(19) United States
(12) Patent Application Publication
(10) Pub. No.: US 2013/0331753 A1
(43) Pub. Date: Dec. 12, 2013

(54) UNIVERSAL KNEE ORTHOSIS DEVICE

(71) Applicants: MARK A. FARROW, Tulsa, OK (US); JOEY ADKINS, Hinckley, OH (US)

(72) Inventors: MARK A. FARROW, Tulsa, OK (US); JOEY ADKINS, Hinckley, OH (US)

(73) Assignee: COMPRESSION SOLUTIONS, INC., Tulsa, OK (US)

(21) Appl. No.: 13/490,179

(22) Filed: Jun. 6, 2012

(51) Int. Cl. A61F 5/00

(52) U.S. Cl. 602/16; 602/13

(57) ABSTRACT

A knee orthosis device contains a bladder capable of sequential or cyclical compression of at least two inflatable chambers. An optional gel pack may be removably affixed to an inside surface of the bladder for providing cold therapy to a user. The wrap is preferably provided with removable and relocatable hinge bars. The hinge bars preferably are provided with conjular pads. The hinge bars may be located with the assistance of locating indicia and installed and affixed within the device and affixed at a desired location on an inside surface of the bladder so that the hinges of the hinge bars are positioned over a transverse axis of a user's knee, thereby ensuring that the knee wrap device of the invention provides a universal fit.

### FIELD OF THE INVENTION

The present invention relates to therapeutic medical devices and more particularly to devices for improving venous blood flow in a patient.

### BACKGROUND OF THE INVENTION

The human knee permits flexion and extension about a transverse axis. The center of the transverse axis of the extension and flexion movements is located where the lateral and medial collateral ligaments and posterior and anterior cruciate ligaments intersect.

A knee orthosis is a device worn to support the knee. The device is worn around the knee to relieve pressure on a part of the knee joint that is affected by surgery, injury, or ailments such as arthritis or osteoarthritis. A knee orthosis device can provide stability needed to perform daily activities. Knee orthosis devices may also assist in knee alignment to help reduce osteoarthritis pain.

A knee orthosis device may be provided with hinges to reduce the amount of stress or pressure being applied to the knee and to support a portion of the weight on a selected side of the knee such as may be desirable for a knee affected by arthritis. Hinged braces can be made with one or two hinges. A hinged brace provides stability during walking, standing, or during the motions of physical therapy exercises and can relieve pain considerably.

### SUMMARY OF THE INVENTION

A knee orthosis device is provided that is capable of providing sequential or cyclical compression of at least two inflatable chambers. The device is provided with hinge bars. In a preferred embodiment, the hinge bars are provided with condyle pads that are positioned against the knee of a user. The hinge bars are preferably removable from the device. In a preferred embodiment, the hinge bars may be positioned within the device and affixed at any desired location within the device to properly align the hinge portion of a hinge bar at the transverse axis of a user's knee to facilitate a universal fit.

The universal fit knee orthosis device includes a bladder for facilitating compression. The bladder preferably defines a perimeter seal, a first chamber divider and a second chamber divider. The first chamber divider and the perimeter portion define a first upper chamber and a portion of a lower chamber The second chamber divider and the perimeter portion define a second upper chamber and portion of the lower chamber. The first chamber divider and the perimeter portion define a first air passage between the first upper chamber and the lower chamber. The second chamber divider and the perimeter portion define a second air passage between the second upper chamber and the lower chamber.

Upper and lower straps are preferably provided for affixing the wrap to a user's knee. A fastener, such as a hook and loop fastener, is provided on the straps to engage an outside surface of the bladder or to engage mating hook and loop portions affixed thereto.

A port preferably protrudes from an outer surface of the bladder and preferably communicates with the lower chamber. The first chamber and second chamber receive air through first and second air passages, respectively. The first chamber and the second chamber will, therefore, inflate slightly before the lower chamber. A fluid inlet line communicates with the port for delivering a fluid, such as air, to an inside of the fluid bladder.

An optional gel pack may be provided for removably affixing to the skin side of the bladder to provide cold therapy to a user's knee. The gel pack is preferably of a complementary size to the bladder for locating between the bladder and a user's skin. In a preferred embodiment, the gel pack is formed with indentations for accommodating a condyle pad when the gel pack is used in combination with a condyle pad equipped hinge bar. The gel pack is preferably secured to the bladder with a plurality of fasteners, such as hook and loop fasteners.

Locating indicia are preferably provided on an exterior of the bladder. Alternatively, a plurality of receiving pockets may be provided to assist in positioning the hinge bars for selectively receiving the hinge bars.

A first hinge bar has a first end for affixing adjacent to the locating indicia or, alternatively, for being received in a desired receiving pocket for facilitating a desired orientation of the hinge bar when installed in the wrap. The first hinge bar has an upper member hingedly affixed to a lower member by a first hinge. The upper member has an upper distal end with an upper distal end fastener. The lower member has a lower distal end with a lower distal end fastener. Ideally, the first hinge bar is positioned to locate the hinge portion at the transverse axis of extension and flexion movements of a user's knee. A condyle pad is provided on the hinge portion to enhance comfort for the patient. Preferably, the condyle pad is rotatably mounted on the hinge bar so that the upper and lower hinge bars can pivot with respect to the condyle pad.

A second hinge bar has a second end for affixing adjacent to said locating indicia or, alternatively, for being received in a desired receiving pocket for facilitating a desired orientation of the hinge bar when installed in the device. The second hinge bar has an upper member hingedly affixed to a lower member by a second hinge. The upper member has an upper distal end with an upper distal end fastener. The lower member has a lower distal end with a lower distal end fastener. Ideally, the second hinge bar is positioned to locate the hinge portion at the transverse axis of extension and flexion movements of a user's knee. A condyle pad is provided on the hinge portion to enhance comfort for the patient. Preferably, the condyle pad is rotatably mounted on the hinge bar so that the upper and lower hinge bars can pivot with respect to the condyle pad.

In a preferred embodiment, the upper distal end fastener and the lower distal end fastener of the first hinge bar and the upper distal end fastener and the lower distal end fastener of the second hinge bar may be affixed to the skin side of the bladder with hook and loop fasteners positioned at any desired location, thereby providing a universal fit for the knee orthosis.

### BRIEF DESCRIPTION OF THE DRAWINGS

FIG. 1A is a side elevation view of the wrap or device of the invention positioned on a patient.

FIG. 1B is a front elevation view of the wrap of FIG. 1A positioned on a patient.

FIG. 2A is a plan view of an outside surface of the wrap of FIG. 1.

FIG. 2B is a plan view of an inside surface or skin side surface of the wrap of FIG. 1.

FIG. 3A is a plan view of an outside surface of a cold pack or gel pack sized for locating within the wrap of FIGS. 2A and 2B.

FIG. 3B is a plan view of an inside surface of a cold pack or gel pack sized for locating within the wrap of FIGS. 2A and 2B.

FIG. 4A is a plan view of an outside of a hinge member for installation in the wrap of FIG. 1.

FIG. 4B is a plan view of an inside of a hinge member for installation in the wrap of FIG. 1.

### DETAILED DESCRIPTION OF THE PREFERRED EMBODIMENTS

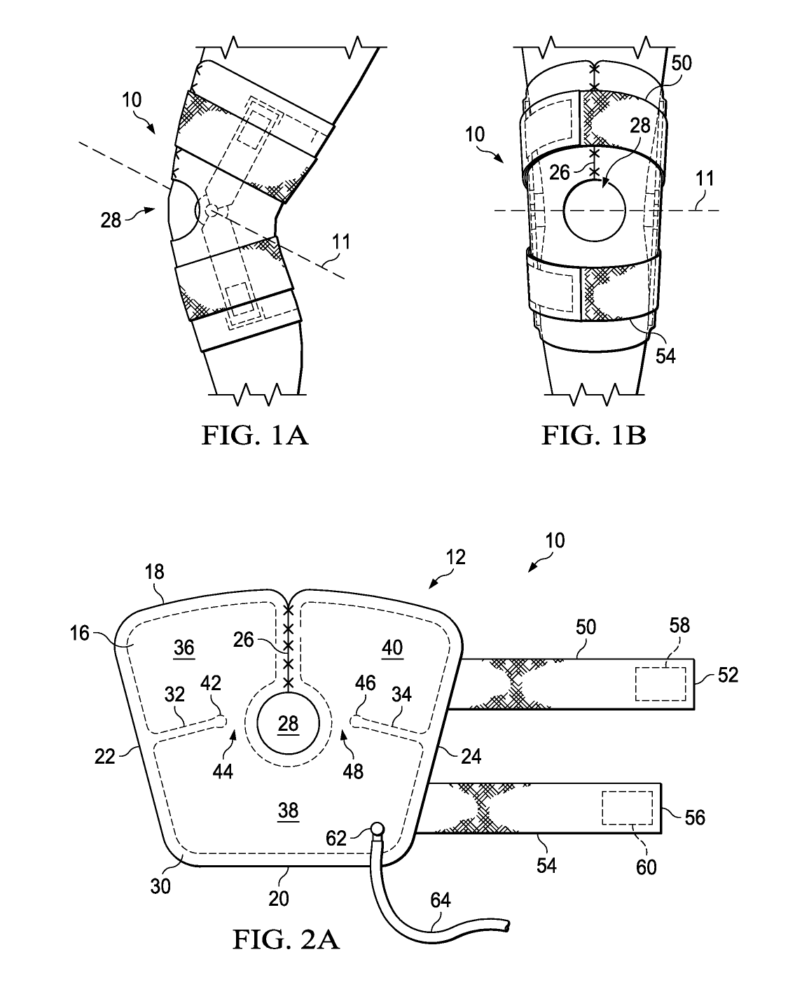

Referring now to the figures, shown is a universal fit knee orthosis designated generally 10. Referring first to FIGS. 1A and 1B, shown is knee orthosis 10 in position on a user's leg. Transverse axis 11 of the user's knee joint is shown for reference.

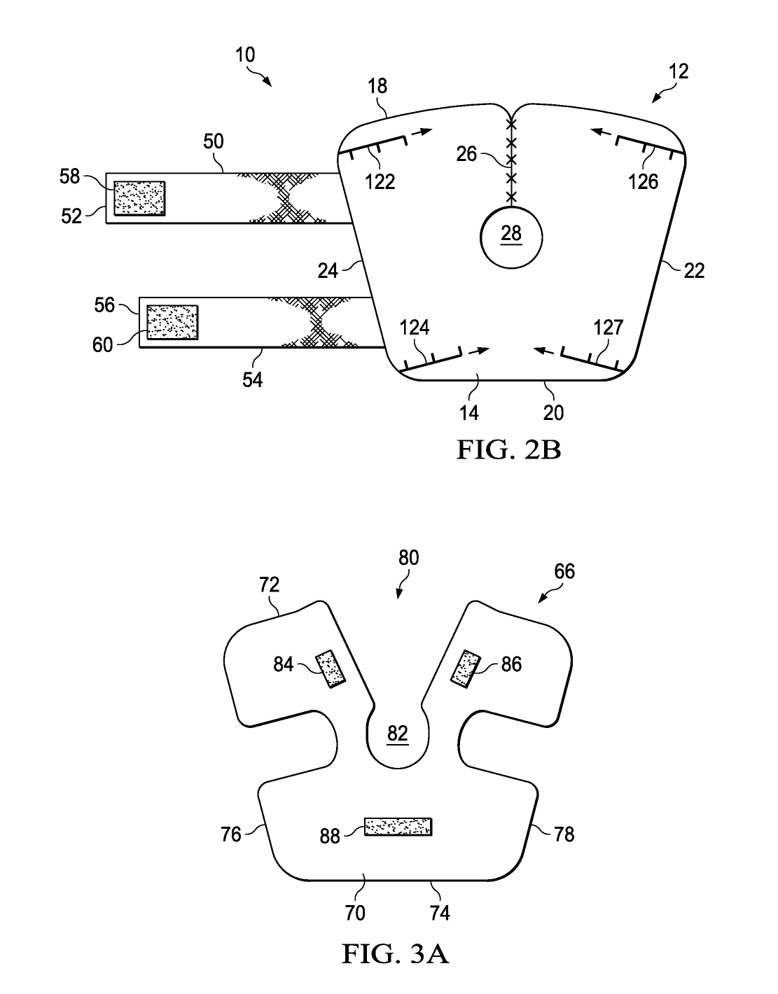

Knee orthosis 10 includes bladder 12 (FIGS. 2A, 2B) having an inside surface or a skin side 14 (FIG. 2B), an outwardly facing side 16 (FIG. 2A), an upper edge 18, a lower edge 20, a first edge 22, and a second edge 24. Upper edge 18 defines a vertical split 26 that terminates in the knee cap orifice 28. Bladder 12 defines a seal having a perimeter portion 30 (FIG. 2A). Perimeter portion 30 preferably has a width of 0.59 inches. Bladder 12 additionally preferably defines a seal having a first chamber line 32 and a second chamber line 34. First chamber line 32 and second chamber line 34 preferably have a width of 0.39 inches. An upper portion of bladder 12 is defined as the portion of bladder 12 above the knee when bladder 12 is positioned adjacent to the knee of a user. In the embodiment of FIGS. 1A-2B, the upper portion of bladder 12 is the portion of bladder 12 above a midpoint of knee cap orifice 28. A lower portion of bladder 12 is defined as the portion of bladder 12 below the knee when bladder 12 is positioned adjacent to the knee of a user. In the embodiment of FIGS. 1A-2B, the lower portion of bladder 12 is the portion of bladder 12 below a midpoint of knee cap orifice 28. Of course, not all embodiments of the invention will necessarily have a knee cap orifice 28. In embodiments not having knee cap orifice 28, upper and lower portions are defined as portions of bladder 12 above and below transverse axis 11 of a user when bladder 12 is positioned adjacent to a knee of a user.

In a preferred embodiment, first chamber line 32 and perimeter portion 30 define first upper chamber 36 and a portion of lower chamber 38.

Second chamber line 34 and perimeter portion 30 preferably also define second upper chamber 40 and a portion of lower chamber 38.

In a preferred embodiment, first chamber line 32 extends inwardly from perimeter portion 30 and terminates at end portion 42. End portion 42 and perimeter portion 30 define first air passage 44. In a preferred embodiment, first air passage 44 is 0.75 inches wide.

Additionally, in a preferred embodiment, second chamber line 34 extends inwardly from perimeter portion 30 and terminates at end portion 46 to define second air passage 48. Second air passage 48 is preferably 0.75 inches wide.

Perimeter portion 30, first chamber line 32, seal chamber line 34, and end portions 42 and 46 are formed by sealing skin side 14 and outwardly facing side 16 together. The seal is preferably created by radio frequency (Rf) welding although other seal forming technologies may also be utilized.

Upper strap 50 extends from one of first edge 22 and second edge 24 of bladder 12. Upper strap 50 has distal end 52. Upper strap 50 preferably has a width of 3 inches. Lower strap 54 extends from one of first edge 22 and second edge 24 of bladder 12. Lower strap 54 has a distal end 56. Lower strap 54 preferably has a width of 3 inches. A first fastener 58 is affixed to distal end 52 of upper strap 50. A second fastener 60 is affixed to distal end 56 of lower strap 54.

Lower strap 54 and upper strap 50 are provided to encircle a user's leg for affixing bladder 12 to a user's leg. First fastener 58 and second fastener 60 are preferably affixable to outwardly facing side 16 of bladder 12.

In a preferred embodiment, port 62 (FIG. 2A) is provided on outwardly facing side 16 of bladder 12. Fluid inlet line 64 is provided to communicate with port 62. Fluid inlet line 64 facilitates the delivery of fluid to an inside of bladder 12.

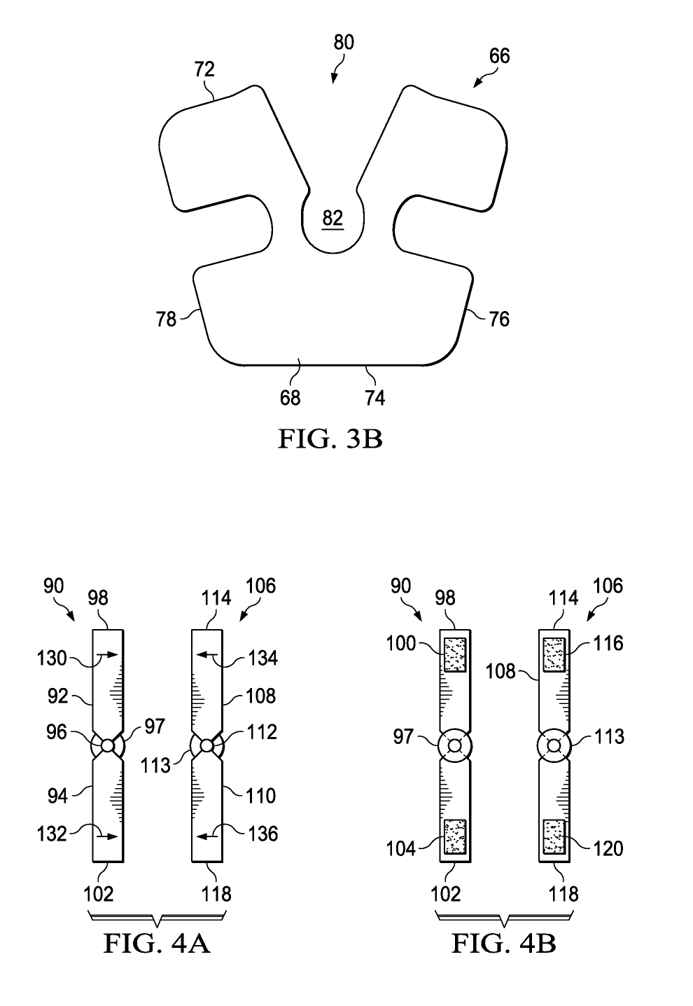

Cold pack or gel pack 66 (FIGS. 3A, 3B) is provided for removably affixing to skin side 14 of bladder 12. Gel pack 66 is preferably of a complimentary size to bladder 12. Gel pack 66 defines an inside surface or a skin side 68 (FIG. 3B), an outside surface or a bladder side 70, upper edge 72, a lower edge 74, a first edge 76, and second edge 78. Upper edge 72 preferably defines vertical split 80 that terminates in knee cap orifice 82.

Referring now to FIG. 3A, first fastener 84, second fastener 86 and third fastener 88 are provided on bladder side 70 of gel pack 66 for removably affixing gel pack 66 to skin side 14 of bladder 12.

First hinge bar 90 (FIGS. 4A, 4B) has an upper member 92 that is hingedly affixed to lower member 94 by first hinge 96. First hinge 96 is preferably covered with condyle pad 97. Upper member 92 has an upper distal end 98. Upper distal end 98 is provided with upper distal end fastener 100 (FIG. 4B). Lower member 94 has a lower distal end 102. Lower distal end 102 has a lower distal end fastener 104.

Second hinge bar 106 has an upper member 108 that is hingedly affixed to lower member 110 by second hinge 112. Second hinge 112 is preferably covered by condyle pad 113. Upper member 108 has an upper distal end 114. Upper distal end 114 has an upper distal end fastener 116. Lower member 110 has a lower distal end 118. Lower distal end 118 has a lower distal end fastener 120.

Upper distal end fastener 100 and lower distal end fastener 104 of first hinge bar 90, and upper distal end fastener 116 and lower distal end fastener 120 of second hinge bar 106 may be affixed to skin side 14 of bladder 12 at any desired location, thereby providing a universal fit for knee orthosis 10.

To assist a user in symmetrical placement of hinge bars 90, 106, if desired, first upper indicia 122 (FIG. 2B) may be provided on skin side 14 of bladder 12. First upper indicia 122 assists a user in locating upper distal end 98 of first hinge bar 90. First lower indicia 124 is provided on skin side 14 of bladder 12. First lower indicia 124 assists a user in locating lower distal end 102 of first hinge bar 90. Additionally, second upper indicia 126 may be provided on skin side 14 of bladder 12 for assisting a user in locating upper distal end 114 of second hinge bar 106. Second lower indicia 127 is provided on skin side 14 of bladder 12. Second lower indicia 127 assists a user in locating lower distal end 118 of second hinge bar 106. Therefore, the wrap of the invention facilitates a universal fit knee orthosis device wherein hinge members 96, 112 of knee wrap hinge bars 90, 106 can be located proximate transverse axis 11 of a user's knee joint. Mating indicia 130, 132, 134, and 136 may be provided on outside of hinge bars 90, 106 to assist a user with a precise placement of hinge bars 90, 106 on bladder 12.

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

What is claimed is:

1. A universal fit knee orthosis device comprising:
a bladder having a skin side, an outwardly facing side, a first edge and a second edge, said bladder for locating adjacent to a knee of a user;
a first hinge bar having an upper member affixed to a lower member by a hinge, said upper member for engaging an upper portion of said bladder, said lower member for engaging a lower portion of said bladder;
wherein said first hinge bar may be affixed to said bladder at any location on said skin side of said bladder, thereby facilitating universal fit of the knee orthosis device.

2. The knee orthosis device of claim 1 further comprising:
a first condyle pad adjacent to said hinge of said first hinge bar.

3. The knee orthosis device of claim 1 further comprising:
a second hinge bar having an upper member affixed to a lower member by a second hinge, said upper member for engaging said upper portion of said bladder above the knee of the user, said lower member for engaging said lower portion of said bladder below the knee of the user.

4. The knee orthosis device of claim 3 further comprising:
a second condyle pad adjacent to said second hinge of said second hinge bar.

5. The knee orthosis device of claim 1 wherein:
said bladder defines a plurality of chambers in communication with one another by air passages.

6. The knee orthosis device of claim 5 wherein:
said plurality of chambers comprise a lower chamber, a first chamber, and a second chamber.

7. The knee orthosis device of claim 1 further comprising:
a strap extending from one of said first edge and said second edge of said bladder for securing said bladder adjacent to the knee of the user.

8. The knee orthosis device of claim 7 further comprising:
a fastener affixed to a distal end of said strap;
wherein said fastener is affixible to said outwardly facing side of said bladder.

9. The knee orthosis device according to claim 1 further comprising:
a port on said outwardly facing side of said bladder.

10. The knee orthosis device according to claim 9 further comprising:
a fluid inlet line in communication with said port, said fluid inlet line for delivering fluid to an inside of said bladder.

11. The knee orthosis device according to claim 1 further comprising:
a gel pack for removably affixing to said skin side of said bladder.

12. The knee orthosis device according to claim 2 wherein:
a gel pack for removable affixing to said skin side of said bladder wherein:
said gel pack defines indentations for accommodating said first condyle pad so that said first condyle pad may contact the knee of the user when said gel pack is utilized.

13. The knee orthosis device according to claim 12 further comprising:
at least one fastener on a bladder side of said gel pack for removably affixing said gel pack to said skin side of said bladder.

14. The knee orthosis device according to claim 1 wherein:
said upper member of said first hinge bar has an upper distal end having an upper distal end fastener and said lower member of said first hinge bar has a lower distal end having a lower distal end fastener.

15. The knee orthosis device according to claim 14 wherein:
said upper distal end fastener and said lower distal end fastener of said first hinge bar may be affixed to said skin side of said bladder at any desired location, thereby providing a universal fit for the knee orthosis device.

16. The knee orthosis device according to claim 15 wherein:
said upper distal end fastener and said lower distal end fastener of said first hinge bar are comprised of a hook or loop fastener for engaging said skin side of said bladder.

17. The knee orthosis device according to claim 1 further comprising:
a first indicia on said skin side of said bladder for assisting the user in locating said upper member of said first hinge bar;
a second indicia on said skin side of said bladder for assisting a user in locating said lower member of said first hinge bar.

18. A method of locating a knee orthosis device on a patient comprising:
positioning a first hinge bar on a skin side of a bladder by attaching an upper member of said first hinge bar to said bladder and by attaching a lower member of said first hinge bar to said bladder;
positioning said bladder and attached first hinge bar adjacent to a knee of a user; and
checking that a hinge of said first hinge bar is positioned proximate a transverse axis of the knee of the user.

19. The method according to claim 19 further comprising the steps of:
removing said bladder;
repositioning said upper member and said lower member as necessary to re-position said hinge such that said hinge is proximate said transverse axis when said bladder is re-positioned on the knee of the user;
re-positioning said bladder and said attached first hinge bar adjacent to the knee of the user.

20. The method of claim 19 wherein said step of repositioning said bladder and said attached first hinge bar further comprises the step of:
locating a first condyle pad adjacent to the knee of the user proximate said transverse axis, wherein said first condyle pad is adjacent to said hinge of said first hinge bar.

21. The method according to claim 18 further comprising the step of:
locating a gel pack between said bladder and the knee of the user.
